(19) United States
(12) Patent Application Publication
(10) Pub. No.: US 2009/0326282 A1
(43) Pub. Date: Dec. 31, 2009

(54) PROCESS FOR THE MANUFACTURE OF BICYCLIC MOLECULES BY COPPER-CATALYSED PHOTOCHEMICAL CYCLISATION

(75) Inventors: Florian Rampf, Hegenheim (FR); Michael Ruther, Langenfeld (DE); Robert Söllner, Leverkusen (DE)

Correspondence Address: LANXESS CORPORATION, 111 RIDC PARK WEST DRIVE, PITTSBURGH, PA, 15275-1112, US

(73) Assignee: SALTIGO GMBH, Langenfeld (DE)

(21) Appl. No.: 11/919,304

(22) PCT Filed: Apr. 21, 2006
(86) PCT No.: PCT/IB2006/001136

§ 371 Date: Sep. 14, 2009

Related U.S. Application Data
(60) Provisional application No. 60/676,024, filed on Apr. 28, 2005.

(51) Int. Cl. C07C 35/23

(52) U.S. Cl. 568/819

(57) ABSTRACT

The present invention relates to a new copper-catalysed photochemnical cyclisation process for the preparation of bicylic molecules useful as intermediates in the manufacture of drugs. In a preferred aspect, the process of the invention may be used to prepare 3-hydroxy-bicyclo[3.2.0]heptane.

This invention relates to a new process for the manufacture of bicylic molecules useful as intermediates in the manufacture of drugs. More specifically, the invention relates to a copper-catalysed photochemical cyclisation process for the manufacture of such molecules. In a preferred aspect, the process of the invention may be used to prepare 3-hydroxy-bicyclo[3.2.0]heptane.

The intramolecular cyclisation of dienes in the presence of ultraviolet light and a copper (I) catalyst has been known for a number of years. Early studies on the intramolecular cyclisation of norbornadiene, for instance, utilised copper(I)chloride and copper(I)bromide (J. Am. Chem. Soc., 1977, 99, 5677). The reaction was reported to proceed in a variety of solvents, such as ethanol, chloroform, tetrahydrofuran and acetonitrile, suggesting that the choice of solvent was not critical.

Subsequent investigations have studied the analogous intramolecular cyclisation of linear 1,6-dienes which provides a convenient synthesis of bicyclo[3.2.0]heptane derivatives (e.g. Tetrahedron Letters, 1978, 9, 821; J. Org. Chem., 1988, 53, 3673; J. Am. Chem. Soc., 1991, 113, 657). These studies have predominantly taught the use of copper(I)triflate, which is preferred due to the weakly coordinating nature of the triflate anion in comparison with halide anions. Such weak coordination facilitates the formation of a complex between the double bonds of the diene and the copper which is a necessary prerequisite for the reaction to proceed. Solvents such as ether, acetone and tetrahydrofuran have been used in combination with medium and high pressure ultraviolet lamps.

In one of these studies (Tetrahedron Letters, 1978, 9, 821), Evers and Mackor demonstrated that a compound of formula (I) (1,6-heptadiene-4-ol, see Scheme 1) could be photocyclised to give a compound of formula (II) (3-hydroxy-bicyclo[3.2.0]heptane) using copper(I)triflate.benzene (CuOTf.benzene) as the catalyst, 1,4-dioxane as the solvent and a low pressure ultraviolet lamp as the light source.

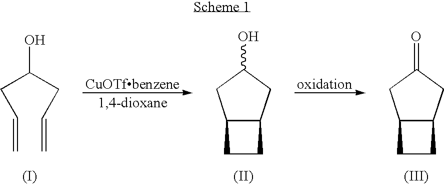

A compound of formula (II) can be easily oxidised to give a compound of formula (III) which is a useful intermediate in the synthesis of (1α,3α,5α)-[3-(aminomethyl)bicyclo[3.2.0]hept-3-yl]acetic acid (IV). Examples of suitable oxidising reagents include chromium(VI) based oxidants such as chromium trioxide and sodium hypochlorite/acetic acid.

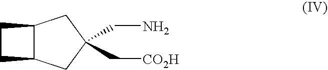

(1α,3α,5α)-[3-(Aminomethyl)bicyclo[3.2.0]hept-3-yl]acetic acid (IV) and its synthesis from ketone (Ill) are disclosed in WO-A-01/28978. This compound is described as being useful in the treatment of epilepsy, pain and other conditions.

Drug molecules must be synthesised in large amounts in order to satisfy worldwide demand and such a synthesis should be efficient and cheap in order to be commercially viable. There therefore exists a need to provide better syntheses of (1α,3α,5α)-[3-(aminomethyl)bicyclo[3.2.0]hept-3-yl]acetic acid (IV) which are shorter or more efficient, especially when carried out on a large scale. A better synthesis of compound (II), which is an important intermediate in the synthesis of compound (IV), would therefore be highly desirable.

Present photochemical routes to compound (II), as described above, involve the use of copper(I) salts such as copper(I)triflate which is sensitive to air and moisture, rather expensive and consequently unsuitable for large scale synthesis.

Such prior art methods are also rather low-yielding and produce unwanted by-products. Surprisingly, we have now found that a more efficient synthesis of compound (II) is provided by the photocyclisation of a compound of formula (I) in the presence of (a) a copper(I) catalyst generated by the in situ reduction of a copper(II) salt or (b) a copper (II) salt alone. The reaction has also been successfully applied to analogues of compound (I).

The invention therefore provides a process for preparation of a compound of formula (V):

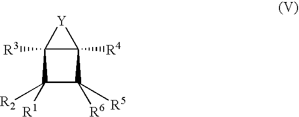

wherein Y is optionally substituted C₂-C₅ alkylene, one of the —CH₂— groups of said C₂-C₅ alkylene being optionally replaced by a sulphur atom, an oxygen atom or an optionally substituted nitrogen atom; and each of R¹, R², R³, R⁴, R⁵ and R⁶ is individually either H, C₁-C₆ alkyl, C₁-C₆ haloalkyl, C₃-C₈ cycloalkyl or C₃-C₈ halocycloalkyl;

comprising the photocyclisation of a compound of formula (VI):

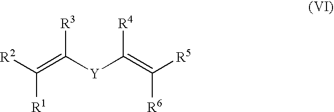

wherein Y, R¹, R², R³, R⁴, R⁵ and R⁶ are as defined above, in the presence of (a) a copper(I) catalyst generated by in situ reduction of a copper(II) salt or (b) a copper(II) salt alone. The preparation is fast, efficient and high-yielding and can easily be scaled up to provide multikilogram quantities of the product.

Halo means fluoro or chloro.

Y is preferably optionally substituted n-propylene, more preferably n-propylene optionally substituted by hydroxy, most preferably —CH₂CH(OH)CH₂—.

R¹, R², R³, R⁴, R⁵ and R⁶ are each preferably H or C₁-C₆ alkyl, most preferably H.

The compound of formula (VI) is most preferably 1,6-heptadiene-4-ol.

Preferred copper(II) salts (for use alone or in conjunction with a reducing agent) are those with poorly coordinating counter-ions such as copper(II)nitrate, copper(II)sulphate and copper(II)triflate, either in the anhydrous or hydrated state. Most preferred is copper(II)sulphate, particularly in the hydrated form.

The preferred reducing agent is a sulphite or hydrogensulphite, particularly sodium hydrogensulphite.

The reaction is carried out in solution in a suitable solvent. The solvent is preferably one which does not appreciably absorb ultraviolet light (particularly light at a wavelength of 254 nM), which is well adapted to large scale synthesis and which can sufficiently solubilise the starting material. Typically the solvent is therefore water or a mixture of water and a water-miscible organic co-solvent such as an alcohol, a diol (e.g. ethane-1,2-diol), a polyol, polyethylene glycol, polypropylene glycol or dimethoxyethane. Preferred solvents are water and a mixture of water with a C₁-C₆ alcohol which have the advantage of being almost UV transparent at 254 nM (unlike, for example, dioxane) and which are non-hazardous when used on an industrial scale (unlike, for example, ethereal solvents). Particularly preferred solvents are water and aqueous ethanol. The use of such aqueous solvents leads to a reaction which is surprisingly fast and high-yielding considering the potential for tight co-ordination between molecules of the solvent and the copper catalyst.

A source of ultraviolet light is required for a photochemical cyclisation. Any ultraviolet lap which has a significant output at a wavelength of from 240 to 280 nM (particularly at 254 nM) can be used, but mercury vapour lamps, particularly low and medium pressure mercury vapour lamps, most particularly low pressure mercury vapour lamps, are preferred. Large scale mercury vapour lamps developed for the treatment of waste or drinking water are particularly suitable for large scale synthesis, low pressure varieties being most preferred. If desired, unwanted wavelengths (particularly wavelengths lower than 230 nM which can lead to unwanted polymerisation) can be filtered out using methods well known in the art.

In a preferred embodiment, the reaction is performed under an inert atmosphere which excludes air, such as an atmosphere of nitrogen or argon.

In a typical procedure, the copper(II) salt and the compound of formula (I) are mixed with the chosen solvent, oxygen is purged from the system and the reducing agent is added (if required). The reaction mixture is then purged again and exposed to ultraviolet light until the reaction is substantially complete. Alternatively, the compound of formula (I) may be added slowly to the reaction medium at the rate at which it is consumed. When the reaction is finished, the product is isolated by conventional methods such as extraction into an organic solvent or distillation.

The invention is illustrated by the following examples.

### EXAMPLE 1

Anhydrous copper sulphate (78 mg) was dissolved in water (300 ml) and 1,6-heptadiene-4-ol (5 g) was added. The mixture was degassed by purging with argon. An aqueous solution of sodium hydrogensulphite (39% w/w, 0.2 ml) was added and the mixture was degassed once more. The reaction mixture was exposed to ultraviolet light (TQ150 Heraeus medium-pressure mercury vapour lamp) for 2.5 hours after which time gas chromatographic analysis of the reaction mixture showed complete conversion of the starting material. The reaction mixture was extracted with dichloromethane and the organic phase was concentrated to yield bicyclo[3.2.0]heptane-3-ol (4.9 g) as a mixture of exo and endo isomers.

### EXAMPLE 2

Anhydrous copper sulphate (78 mg) was dissolved in water (300 ml) and 4-methyl-1,6-heptadiene-4-ol (5 g) was added. The mixture was degassed by purging with argon. An aqueous solution of sodium hydrogensulphite (39% w/w, 0.2 ml) was added and the mixture was degassed once more. The reaction mixture was exposed to ultraviolet light (TQ150 Heraeus medium-pressure mercury vapour lamp) for 2.5 hours after which time gas chromatographic analysis of the reaction mixture showed complete conversion of the starting material. The reaction mixture was extracted with dichloromethane and the organic phase was concentrated to yield cis-2-methylbicyclo[3.2.0]heptane-2-ol (4.9 g).

### EXAMPLE 3

Anhydrous copper sulphate (711 mg) was dissolved in a mixture of water (100 ml) and ethanol (207 ml) and 1,6-heptadiene-4-ol (20 g) was added. The mixture was degassed by purging with argon. An aqueous solution of sodium hydrogensulphite (39% w/w, 0.2 ml) was added and the mixture was degassed once more. The reaction mixture was exposed to ultraviolet light (TQ150 Heraeus medium-pressure mercury vapour lamp) for 24 hours after which time gas chromatographic analysis of the reaction mixture showed 90% conversion of the starting material. The reaction mixture was extracted with dichloromethane and the organic phase was concentrated to yield bicyclo[3.2.0]heptane-3-ol (18.4 g).

### EXAMPLE 4

1,6-Heptadiene-4-ol was converted to bicyclo[3.2.0]heptane-3-ol under various conditions as summarised in Table 1. In all cases 20 g of 1,6-heptadiene-4-ol was dissolved in 300 ml of solvent and irradiated with-a 150 W Heraeus TQ 150 medium pressure Hg vapour lamp for 24 hours in the presence of the indicated catalyst. In the case of experiments 1 to 3, precipitated copper salts were filtered off prior to exposure to UV light. The reaction mixtures were analysed by GC. As very small amounts of side products were formed, the yield of product reflects the reaction rate under the chosen conditions.

TABLE 1

| | | | | Yield | Yield |
|---|---|---|---|---|---|
| | | catalyst loading | | starting | product |
| | Catalyst | mol % | Solvent | material (%) | (%) |
| 1 | C | 0.5 | EtOH/H₂O | 41 | 59 |
| 2 | C | 2.5 | EtOH/H₂O | 36 | 57 |
| 3 | C | 5.0 | EtOH/H₂O | 49 | 51 |
| 4 | C | 2.5 | EtOH/H₂O | 28 | 72 |
| 5 | B | 0.5 | EtOH/H₂O | 31 | 67 |
| 6 | B | 2.5 | EtOH/H₂O | 13 | 87 |
| 7 | C | 2.5 | Glycol/H₂O | 5 | 93 |
| 8 | C | 2.5 | H₂O | 1 | 98 |

B = copper(II)triflate
C = copper(II)sulphate
EtOH/H₂O = 2:1 mixture by weight of ethanol:water
Glycol/H₂O = 2:1 mixture by weight of ethane-1,2-diol:water

1. A process for preparation of a compound of formula (V): 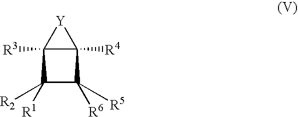
wherein Y is optionally substituted C₂-C₅ alkylene, one of the —CH₂— groups of said C₂-C₅ alkylene being optionally replaced by a sulphur atom, an oxygen atom or an optionally substituted nitrogen atom; and each of R¹, R², R³, R⁴, R⁵ and R⁶ is individually either H, C₁-C₆ alkyl, C₁-C₆ haloalkyl, C₃-C₈ cycloalkyl or C₃-C₈ halocycloalkyl;
comprising the photocyclisation of a compound of formula (VI): 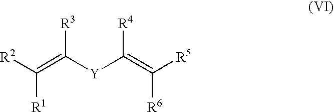
wherein Y, R¹, R², R³ , R⁴, R⁵ and R⁶ are as defined above, in the presence of (a) a copper(I) catalyst generated by in situ reduction of a copper(II) salt or (b) a copper(II) salt alone.

2. A process as claimed in claim 1 wherein the compound of formula (VI) is 1,6-heptadiene-4-ol.

3. A process as claimed in claim 1 or claim 2 wherein the copper(II) salt is copper(II)sulphate.

4. A process as claimed in any preceding claim wherein the reducing agent is sodium hydrogensulphite.

5. A process as claimed in any preceding claim wherein the reaction is carried out in a solvent selected from water, a water/C₁-C₆ alkanol mixture and a water/C₁-C₆alkanediol mixture.

6. A process as claimed in any preceding claim wherein the photocyclisation is promoted using a low pressure mercury vapour lamp.
